(19) United States
(12) Patent Application Publication
(10) Pub. No.: US 2022/0017850 A1
(43) Pub. Date: Jan. 20, 2022

(54) SYSTEM, APPARATUS AND METHOD FOR THE PRODUCTION OF CELLS AND/OR CELL PRODUCTS

(71) Applicant: UNIVERCELLS TECHNOLOGIES SA, Nivelles (BE)

(72) Inventor: José Castillo, Brussels (BE)

(21) Appl. No.: 17/360,563

(22) Filed: Jun. 28, 2021

Related U.S. Application Data
(63) Continuation of application No. 15/549,494, filed on Aug. 8, 2017, now abandoned.

(30) Foreign Application Priority Data
Feb. 9, 2015 (BE) BE2015/5064
Feb. 9, 2015 (EP) PCT/EP2015/052633

(51) Int. Cl. C12M 1/36; C12M 1/34; C12M 1/00; C12M 3/00; C12M 3/04; C12M 1/12

(52) U.S. Cl. CPC C12M 41/48; C12M 41/34; C12M 41/40; C12M 47/02; C12M 23/54; C12M 23/40; C12M 27/10; C12M 41/26; C12M 23/44; C12M 29/26; C12M 41/30; C12M 47/16; C12M 23/50; C12M 37/00; C12M 41/00; C12M 41/12

(57) ABSTRACT

The present invention provides a system and a method for the production of cells and/or cell products. The system comprises at least one cell culture unit comprising at least one bioreactor for culturing cells, at least one technical control unit for controlling a cell growth parameters, said technical control unit is at least fluidly connected to the cell culture unit, and at least one air treatment unit for treating ambient air, said air treatment unit is fluidly connected to the cell culture unit. The system is characterized in that the system is autonomous and the bioreactor total volume is at most 1000L.

This application is a continuation of Ser. No. 15/549,494, the disclosure of which is incorporated by reference.

### TECHNICAL FIELD

The invention pertains to methods and systems for the production of cells and/or cell products, such as viruses, proteins or peptides.

### BACKGROUND

With the increased use of cells and cells products, such as viruses, proteins and peptides, in clinical diagnostics and therapy, the need has arisen for more efficient, rapid, sterile production and purification methods.

Conventional approaches and tools for manufacturing cells or cell based products typically involve numerous manual manipulations that are subject to variations even when conducted by skilled technicians. Small quantities of cell-secreted product are produced in different ways. T-flasks, roller bottles, stirred bottles or cell bags are manual methods using incubators or warm-rooms to provide environments for cell growth and production. These methods are very labor intensive, subject to mistakes and difficult for large-scale production.

Production of cells and/or cell secreted products can be achieved using classical stirred tank bioreactors or “special” bioreactors (fibers, microfibers, hollow fiber, ceramic matrix, fluidizer bed, fixed bed, etc.). The systems currently available are general purpose in nature and require considerable time from trained operators to setup, load, flush, inoculate, run, harvest, and unload.

Prior art systems and techniques use a large-scale set-up wherein cells are being grown in batch bioreactors of e.g. 10000 liters (L). Said large-scale set-up systems are not suitable to be placed in a regular laboratory. Moreover, the large-scale set-up systems of the prior art require specific handling and specific installation such as specific lines for providing gas and/or medium to the bioreactor. Indeed, after a cultivation period, the cells and/or cell products of the batch are harvested within about 8 hours. Hereby, the 10000 L of suspension is clarified, the medium is exchanged (cell-culture medium replaced by buffer medium) by diafiltration, and the compounds are separated or purified by chromatography. A further filtration step may follow.

A disadvantage of the prior art techniques include the use of a big filter, a large volume of buffer medium and a considerable volume of purified water. These volumes represent a considerable cost in terms of purified water production and water storage. Another major disadvantage is the yield loss in the clarification step which is an essential step of these large-scale set-up systems for obtaining a diafiltration which is efficient enough to exchange the cell-culture medium within the limit of 8 hours.

Another drawback of the systems of the prior art is the large investments required in terms of necessary installations and space but also in terms of necessary material to produce the desired cells and/or cell products. In addition, the necessary input of energy weighs tremendously on the required budget. The required huge investments restrain development in the field, not only in the US and Europe but also in the developing countries.

It is the aim of the current invention to provide methods and systems for the production of cells and/or cell products which overcome at least part of the above mentioned drawbacks and disadvantages. One object of the invention is to provide automated and integrated methods and systems for the production of cells and/or cell products. Another object of the invention is to provide small-scale set-up and autonomous systems for the production of cells and/or cell products.

### SUMMARY OF THE INVENTION

In a first aspect, the present invention provides a system for the production of cells and/or cell products. The system comprises at least one cell culture unit comprising at least one bioreactor for culturing cells, at least one technical control unit for controlling cell growth parameters, said technical control unit is at least fluidly connected to the cell culture unit, and least one air treatment unit for treating ambient air, said air treatment unit is fluidly connected to the cell culture unit. The system is autonomous and the bioreactor total volume is at most 1000 L.

In a preferred embodiment, the system optionally comprises at least one downstream unit which is fluidly connected to the cell culture unit, said downstream unit comprises pluggable means selected from the group comprising at least one filtration means, at least one harvest means, at least one dialysis means, at least one biomolecules purification means and at least one protein concentration unit or any combination thereof.

In a second aspect, the present invention provides an integrated automated method for the production of cells and/or cell products. The method comprises the steps of culturing cells in at least one bioreactor which is fluidly connected to a culture medium reservoir, said bioreactor being contained in a cell culture unit; providing a mixture of at least two gases to the bioreactor; and providing sterile ambient air into the cell culture unit; wherein the bioreactor total volume is at most 1000 L.

The system and/or the method of the present invention do not require the use of a considerable number of large instruments such as large bioreactors. This is due to the small-scale set-up of the system and to the use of small size bioreactor. Another advantage is the autonomy of the system. The latter should only be connected to an external cell culture medium reservoir. No other specific tubing and connections are required such as gas lines. This simplifies the installation of the system which thereby can be installed and used in a regular laboratory wherein only a plug for the system is required. The installation costs are also considerably reduced.

Furthermore, the present method and system are devoid of manual handling, thereby considerably reducing contamination risk. In addition the autonomous system of the invention allows performing the full process under high biosafety circumstances thanks to the air treatment unit.

The systems and methods of the invention also allow rapid production of cells and/or cell products using significantly smaller equipment compared to the prior art systems and methods. Another advantage is to provide for high yield cells and/or cell products production compared to the methods and the systems of the prior art thereby reducing costs of the final product. The present invention provides cheaper fully-automated and integrated systems, which cost is at least 5 to 6 times less than the usual large-scale set-up systems. This eventually results in a lower investment and production cost, which is a considerable advantage.

### DESCRIPTION OF FIGURES

FIG. 1 shows an embodiment of the system of the invention.

FIG. 2 shows an embodiment of the system of the invention wherein the cell culture unit is connected to a downstream unit comprising filtration means and purification means.

### DETAILED DESCRIPTION OF THE INVENTION

The present invention concerns methods, apparatuses and systems for the production of cells and/or cell products or biomolecules such as viruses, proteins or peptides. The invention specifically aims to provide a small scale system implementable in a laboratory. Said system and method have an optimal efficiency in terms of input of material and products output. The current invention thereto aims to provide a fully integrated and automated methodology and system for the production of cells and/or biomolecules. By “proteins or peptides” and “cells and/or biomolecules” reference is made to antibodies as well as antigens.

Unless otherwise defined, all terms used in disclosing the invention, including technical and scientific terms, have the meaning as commonly understood by one of ordinary skill in the art to which this invention belongs. By means of further guidance, term definitions are included to better appreciate the teaching of the present invention.

As used herein, the following terms have the following meanings: “A”, “an”, and “the” as used herein refers to both singular and plural referents unless the context clearly dictates otherwise. By way of example, “a compartment” refers to one or more than one compartment.

“About” as used herein referring to a measurable value such as a parameter, an amount, a temporal duration, and the like, is meant to encompass variations of +/−20% or less, preferably +/−10% or less, more preferably +/−5% or less, even more preferably +/−1% or less, and still more preferably +/−0.1% or less of and from the specified value, in so far such variations are appropriate to perform in the disclosed invention. However, it is to be understood that the value to which the modifier “about” refers is itself also specifically disclosed.

“Comprise,” “comprising,” and “comprises” and “comprised of” as used herein are synonymous with “include”, “including”, “includes” or “contain”, “containing”, “contains” and are inclusive or open-ended terms that specifies the presence of what follows e.g. component and do not exclude or preclude the presence of additional, non-recited components, features, element, members, steps, known in the art or disclosed therein.

The recitation of numerical ranges by endpoints includes all numbers and fractions subsumed within that range, as well as the recited endpoints.

The expression “% by weight” (weight percent), here and throughout the description unless otherwise defined, refers to the relative weight of the respective component based on the overall weight of the formulation.

The system and the method of the invention will now be described with reference to the accompanying figures.

In a first aspect, the present invention provides a system for the production of cells and/or cell products, comprising:

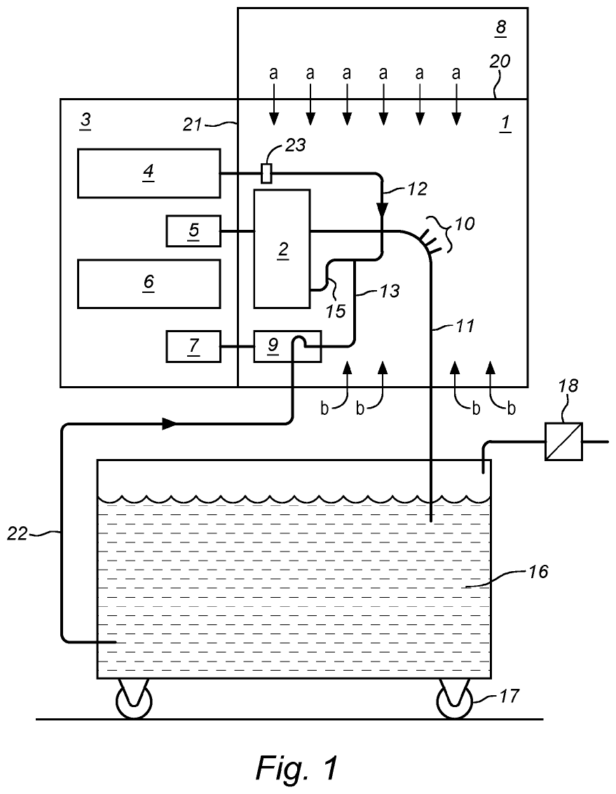

at least one cell culture unit 1 comprising at least one bioreactor 2 for culturing cells. said bioreactor may be attached to the system in a fixed manner, or may be removably attached to the system,
at least one technical control unit 3 for controlling a cell growth parameters, said technical control unit is at least fluidly connected to the cell culture unit 1, and
at least one air treatment unit 8 for treating ambient air, said air treatment unit is fluidly connected to the cell culture unit 1 (FIG. 1). The cell culture unit 1 and the technical control unit 3 have at least one common wall 21. Also, the cell culture unit 1 and the air treatment unit 8 have at least one common wall (20 in FIG. 1).

The system is characterized in that it is autonomous and the bioreactor total volume is at most 1000 L. By bioreactor total volume reference is made to the total liquid volume that can be introduced in the bioreactor, thereby totally filling the bioreactor. By autonomous reference is made to a system which is devoid of external connections for gas supply and/or external connections for system sterilization and/or external connections for taking samples from the culture medium comprised in the bioreactor.

The system is autonomous as it is devoid of connections to external source of oxygen and/or air and/or CO₂. The system requires power supply to function properly. This is advantageous as the system does not require heavy installations and/or tubing which ensure connection to oxygen and/or air and/or CO₂. The system is therefore easily transportable and can be implemented in any room in which at least a power supply is provided.

In a preferred embodiment, the bioreactor total volume is at most 980 L, at most 960 L, at most 940 L, at most 920 L, at most 900 L, at most 880 L, at most 860 L, at most 840 L, at most 820 L, at most 800 L, at most 780 L, at most 760 L, at most 740 L, at most 720 L, at most 700 L, at most 680 L, at most 660 L, at most 640 L, at most 620 L, at most 600 L, at most 580 L, at most 560 L, at most 540 L, at most 520 L, at most 500 L, at most 490 L, at most 480 L, at most 450 L, at most 420 L, at most 400 L, at most 380 L, at most 350 L, at most 340 L, at most 330 L, at most 320 L, at most 310 L, at most 300 L, at most 290 L, at most 280 L, at most 270 L, at most 260 L, at most 250 L, at most 240 L, at most 230 L, at most 220 L, at most 210 L, at most 200 L, at most 190 L, at most 180 L, at most 170 L, at most 160 L, at most 150 L, at most 140 L, at most 130 L, at most 120 L or at most 100 L, or any value comprised in between the aforementioned values.

In a preferred embodiment, the bioreactor total volume is at least 0.5 L, preferably at least 1.5 L, preferably at least 3 L, more preferably at least 5 L, even more preferably at least 10 L, most preferably at least 20 L, even most preferably at least 30 L. Preferably, the bioreactor total volume is at least 40 L, at least 50 L, at least 60 L, at least 70 L, at least 80 L, at least 90 L or any value comprised in between the aforementioned values. The bioreactor total volume and the bioreactor itself are small compared to the conventional bioreactors used for cell culture. This is advantageous in terms of required space for the system and for ease of use.

The cell culture unit provides for production of cells and cell derived products in a closed, self-sufficient environment. Said unit may comprise at least one bioreactor for cells and/or their products expansion with minimal need for technician interaction. Said bioreactor may be attached to the system in a fixed manner, or may be removably attached to said system.

In a preferred embodiment, the technical control unit 3 comprises at least one motion means 5 for moving the bioreactor 2, said motion means 5 is mechanically and/or magnetically connected to the bioreactor 2 and provides a movement selected from movements from right to left, up to down, rotating along a horizontal axis, rotating along a vertical axis, a rocking motion along a tilted or inclined horizontal axis of the bioreactor or any combination thereof. Said movement or motioning might be performed in a continuous or discontinuous mode. Preferably, for rotational movement, the bioreactor is mounted to the wall 21 which is common to the cell culture unit 1 and the technical control unit 3 (FIG. 1).

Cells require oxygen during their growth phase in order to have an optimal growth. The bioreactor can be subject to motioning, thereby increasing oxygen transfer by a factor of at least 10 compared to conventional systems and methods and ensuring gas equilibrium in said bioreactor. Operation of the bioreactor at gas equilibrium is hence achieved. This on its turn increases cell growth, which has a positive impact on biomolecules production. This allows to run cultures in a bioreactor which is devoid of sensors thereby providing a simple and less complicated bioreactor installation as well as a straightforward and simple methodology compared to the bioreactors and methodologies of the prior art. In addition, the use of a bioreactor devoid of sensors provides for a considerable decrease of contamination risk. Furthermore, sensor failure is no longer an issue, and repairs which were needed in prior art systems due to said sensor failure are no longer required, leading to a high reduction in operation and personnel costs.

Motioning the bioreactor further improves cells harvesting. Indeed, harvesting cells from a carriers-containing bioreactor, such as fibers or microfibers bioreactors has been difficult to accomplish. Typically, cells are sticky and attach themselves to the carriers or to other cells and form clusters. Motioning the bioreactor forces the cells free thereby providing increased efficiency of cell harvest at high cell viabilities without the use of chemical or enzymatic release additives. The bioreactor may have a rigid or a non-rigid outer body. Rigid outer body allows for the bioreactor case to be flexed causing microfiber movement. This movement enhances the release of cells that have attached inside the bioreactor matrix.

In a preferred embodiment, the technical control unit 3 comprises at least one supply means 7,9 connected to the bioreactor 2 and to a culture medium reservoir 16 for providing the bioreactor 2 with culture medium (FIG. 1). Said culture medium reservoir 16 is positioned outside the system and may be provided with wheels 17 for an easy transport of said reservoir. The culture medium reservoir 16 might be connected to a sterile filter 18 for venting. Said culture medium reservoir can be provided with at least one heat and/or cooling means in order for the culture medium to be supplied at the desired temperature to the bioreactor 2.

Preferably, the technical control unit 3 comprises more than one supply means for providing the bioreactor 2 with any other required element for the cell growth. Said elements are contained in at least one external reservoir and selected from the lists comprising additives, cells, pH adjusting solutions or any combination thereof. The technical control unit might comprise 2, 3, 4, 5 or more supply means.

The supply means 7,9 might be a peristaltic pump whereby both the motor and the pump itself—also herein called the pump head—are positioned in the technical control unit 3. Both the motor and the pump head might also be positioned in the cell culture unit 1. Preferably, the pump motor 7 is positioned in the technical control unit 3 while the pump head 9 is positioned in the cell culture unit 1 (FIG. 1). This allows gaining space in the cell culture unit and to avoid having the moving part of the pump in said unit thereby minimizing the presence of particles.

If the pump is positioned in the cell culture unit 1, then a pipe or tube 13 is provided between said pump and the bioreactor 2 thereby fluidly connecting them to each other. It is to be understood that the supply means 7,9 is fluidly connected to the culture medium reservoir by at least one tube 22 or any equivalent means known to the person skilled in the art. This allows supplying the bioreactor 2 with culture medium (FIG. 1). In this case, the pipe or tube 13 provided between said pump and the bioreactor 2 is the same as the tube 22 fluidly connecting the supply means 7,9 to the culture medium reservoir (FIG. 1). The supply means 7,9 is also fluidly connected to the bioreactor 2 by at least on inlet pipe 15 or any equivalent means known to the person skilled in the art.

Preferably, the culture medium is pre-heated to a temperature of between 25° C. to 37° C. and/or mixed prior to transfer to the bioreactor. This ensures that the cells will not perceive a cold-shock when being contacted with new medium—which would negatively affect their growth—as well as ensure that all nutrients in the medium are mixed and present in the required amounts. Said pre-heating and/or mixing is performed in the culture medium reservoir 16 using at least one heating and/or cooling means and at least one mixing means (not shown). The mixing means might be accommodated inside or outside the reservoir 16. The heating and/or cooling means could be positioned between the reservoir 16 and the bioreactor 2. The culture medium can be a liquid comprising a well-defined mixture of salts, amino acids, vitamins and one or more protein growth factors. The culture medium serves to deliver nutrients to the cell and conversely, to remove or prevent a toxic build-up of metabolic waste.

In a preferred embodiment, the technical control unit 3 comprises at least one measurement means 6 for measuring a plurality of cell culture parameters. Said parameters are selected from, but are not limited to, oxygen level, pH, temperature and cell biomass. Preferably, the measurement means comprise at least one transmitter and/or at least one sensor or any other means known to the person skilled in the art. Said sensors might be optical sensors, light based sensors, radio-frequency, WiFi based sensors or any other sensors known to the person skilled in the art. Preferably, sensors which do not require physical connection to the bioreactor or any other element of the system are used.

Preferably, the bioreactor itself is devoid of sensors. Said sensors might be provided outside the bioreactor. This providing a simple and less complicated bioreactor installation as well as a straightforward and simple methodology compared to the bioreactors and methodologies of the prior art. In addition, the use of a bioreactor devoid of sensors provides for a considerable decrease of contamination risk. In another preferred embodiment, the bioreactor might be provided with at least on sensor for measuring at least one of the above mentioned parameters.

In a preferred embodiment, the technical control unit 3 comprises at least one gas production means 4 which is fluidly connected to the bioreactor 2, said gas production means 4 comprises at least one gas mixing device (not shown) for mixing at least two different gases (FIG. 1). The gas production means 4 further comprises at least one oxygen (O₂) production means (not shown) for production of O₂. Said O₂ is preferably produced via enrichment from air using an oxygen concentrator for instance. Said O₂ production might be performed using zeolites comprised in the gas production means 4. The gas production means 4 further comprises at least one CO₂ production means (not shown) for production of CO₂. Said CO₂ production means might be single-use aluminum cans or any other means known to the person skilled in the art. Preferably said CO₂ production is performed at low pressure. Preferably, the gas production means 4 is provided with air pumping means for pumping air directly from the outside environment of the system. Said air pumping means might comprise at least one pipe having one end connected to the outside environment or atmosphere such as a laboratory atmosphere. For instance, if the system is placed in a laboratory, then the gas production means 4 is capable of pumping air from said laboratory. The cell culturing unit 1 is also equipped with means for pumping air directly from the cell culturing unit itself.

Preferably, the gas production means 4 comprises at least one oxygen (02) production means, at least one air production means, at least one CO₂ management means and at least one gas mixing device. The latter mixes different gases according to predetermined ratios. The gas production means is preferably connected to at least one sterile filter for filtering gases prior feeding to the bioreactor.

In a preferred embodiment, the gas mixing device mixes the gases (at least O₂ and CO₂) produced by the gas production means 4 thereby obtaining a pre-mixed gas. The obtained pre-mixed gas is supplied to the bioreactor 2 using one single gas supply line 12. This further simplifies the set-up of the system.

Gas such as pure oxygen or a gaseous mixture comprising oxygen is equally provided through the bioreactor inlet. Oxygen is an essential requirement for the normal growth of mammalian cells. By preference, said gas or gaseous mixture is supplied under pressure. In an embodiment, cells will be exposed to dissolved oxygen concentrations of 300 μM or less (160 mmHg partial pressure), by preference less than 200 μM, most preferably between 20 and 150 μM.

In a preferred embodiment, gas or gaseous mixture and culture medium will be intermixed prior being supplied to the bioreactor. Hence, the mix of gas or gaseous mixture and culture medium are supplied to through one supply line (15 in FIG. 1). This gives as an advantage that a cell medium with optimal oxygen concentration is provided directly to the cells. In a further preferred embodiment, said gas or gaseous mixture is chosen from air or oxygen. By preference, air is being used. Air is to be seen as a gaseous mixture, comprising approximately 78% of nitrogen, 21% of oxygen and argon and carbon dioxide. Supply of air instead of pure oxygen or oxygen enriched atmospheres has as an advantage that the system employing the method can be omitted of supplying units of highly concentrated oxygen, which may otherwise imply a fire or explosion hazard. These risks are also minimized and even inexistent using the system of the invention as said system is provided with its own 02 production means.

The low solubility of oxygen in aqueous medium (such as a cell culture medium) relative to its rate of consumption causes its rate of supply to be the limiting factor for cell growth. Generally, the oxygen transfer rate (OTR) in a fermentor or bioreactor is described by:

OTR=KLa(C_{gas}−C_{liq}),

Whereby OTR=oxygen transfer rate in μmol O₂|−1 h−1;
KLa=is the oxygen transfer coefficient in h−1;
Cgas=gas-phase O₂ (equilibrium) concentration in μM;
Cliq=liquid phase O₂ concentration in μM

By preference, the oxygen transfer coefficient (KLa) in the current method is at least 20 h⁻¹, preferably at least 30 h⁻¹, more preferably at least 35 h⁻¹. Said oxygen transfer coefficient is at most 100 h⁻¹, preferably at most 50 h⁻¹, more preferably at most 40 h⁻¹.

A high oxygen transfer coefficient and therefore also high OTR will have a positive influence on the cell growth/health and hence the yield of the desired end product. It was found by the inventors that an oxygen transfer coefficient as defined above is particularly beneficial in terms of product yield, even when making use of a rather small amount of cell starter culture.

Ina preferred embodiment, the gas supply line is different from medium supply line. The gas supply line might also be the same as the medium supply line as shown in FIG. 1. In said figure, the medium supply line 13 and the gas supply line 12 supply medium and gas to the same inlet pipe 15 wherein the medium and the gas will be mixed together. This minimizes contamination risks and provided with an easy connection and disconnection system of the bioreactor from the cell culture unit, thereby simplifying its separation from said if the bioreactor needs to be replaced for instance.

In a preferred embodiment, the technical control unit 3 and/or cell culture unit 1 comprise at least one sterile filter 23. Said filter is fluidly connected to the gas production means 4 and to the bioreactor, preferably at the gas supply line 12 as shown in FIG. 1. Thereby, the gaseous mixture flowing through the gas supply line 12 is passed through said sterile filter.

In a preferred embodiment, the air treatment unit 8 comprises at least one sterilization means for providing sterile air to the cell culture unit. Said sterile air is preferably provided to the cell culture unit 1 in a laminar flow (arrows a in FIG. 1). Said sterilization means might be a heating, ventilation and air conditioning system (HVAC system). Said sterilization system allows sterile connections for a plurality of operation that are usually performed in a laminar flow hood. Said operations comprise but are not limited to seeding of cells, viral infection, feeding culture media and/or additives and culture medium sampling. Said culture medium may be supplemented with grown cells and/or biomolecules, from the bioreactor. The sterilization means of the air treatment unit might comprise at least one High-efficiency particulate arrestance (HEPA) filter.

Using conventional incubators, bioreactors should be manipulated and/or moved from one place to another for performing aseptic operations or steps. Using the system and/or the method of the invention, aseptic environment is provided thereby allowing aseptic operations or steps to be carried out on-site during cell culture without manipulation or displacement of the bioreactor itself. Operational and/or contamination risk are thereby considerably reduced. Moreover, culture process is facilitated.

Supplemented culture medium, also herein called supplemented medium, refers to the supernatant of the bioreactor which might comprise culture medium and/or cultured cells and/or their products. The supernatant of the bioreactor might be devoid of cells and/or their products. Cells product refers to biomolecules such as proteins, peptides, produced by the cells and/or any other cell biomolecules derived from cell lysis such as cell membranes.

In a preferred embodiment, the system further comprises at least one programmable controller which is electromechanically connected to the system for controlling and/or monitoring its functioning. The programmable controller is provided with an algorithm which sends instructions to the different units of the system thereby ensuring its functioning for cell and/or cells production. The operator initiates the culture process through a user interface such as a touch screen interface. Said interface might be provided on the system itself or at a distance from said system.

In a preferred embodiment, a predetermined temperature is maintained constant inside the cell culture unit 1. Said predetermined temperature is of about 37° C. Preferably, a predetermined pressure is also maintained constant inside the cell culture unit. The cell culture unit 1 acts similarly to a laminar flow hood thereby allowing sampling of air for particle counting and bio-burden evaluation. The cell culture unit is capable of withdrawing air from the system's outside environment as shown by arrow b in FIG. 1. Said constant pressure allows the manipulation of Biosafety Level 2 microorganisms such as viruses.

The bioreactor used in the method and/or the system of the invention can be any type of bioreactor. The used bioreactor preferably provides a culture surface of at least 0.5 square meters m² per liter of bioreactor. Said bioreactor is preferably a perfusion bioreactor. The bioreactor comprises at least one cells entrapment system or carriers selected from the list comprising fibers, microfibers, hollow microfibers, hollow filter, tangential flow filter, settler, microcarriers, microcarriers containing stirred vessels or any combination thereof. Said carriers provide for an excellent substrate for the cells to grow on.

Preferably, the bioreactor is provided with at least one inlet for the introduction of gas and/or culture medium and at least one outlet for the collection of the culture product and/or the medium contained in the bioreactor. At least one in-tubing is provided for fluidly connecting the bioreactor, via its inlet, to a culture medium tank and/or a gaseous source. At least one out-tubing is provided for fluidly connecting the bioreactor, via its outlet, to a downstream unit and/or any other device.

Preferably, the carriers present in the bioreactor provide a cell growth surface of at least 10 square meters (m²), preferably at least 1000, more preferably at least 1200 m², more preferably at least 1500 m², most preferably at least 1800 m². More preferably, the carriers present in the bioreactor provide a cell growth surface of at least 3 m² per L of bioreactor, preferably at least 4 m², more preferably at least 5 m², even more preferably at least 6 m², most preferably at least 7 m². The carriers might also provide a cell growth surface of at least 8 m² per L of bioreactor, preferably at least 9 m², more preferably at least 10 m², even more preferably at least 11 m², most preferably at least 12 m² per L of bioreactor or any value comprised in between the aforementioned values. The cell growth surface of the bioreactor is also called herein bioreactor expression volume.

The carriers provide a cell growth surface of at most 3000 m², preferably at most 2800 m², more preferably at most 2500 m², even more preferably at most 2200 m², most preferably at most 2000 m². Preferably, the carriers present in the bioreactor provide a cell growth surface of at most 30 m² per L of bioreactor, preferably at most 26 m², more preferably at most 24 m², even more preferably at most 20 m², most preferably at most 19 m². The carriers might also provide a cell growth surface of at most 18 m² per L of bioreactor, preferably at most 17 m², more preferably at most 16 m², even more preferably at most 15 m², most preferably at most 14 m² per L of bioreactor or any value comprised in between the aforementioned values.

The combination of carriers and motioning of the bioreactor significantly increases the oxygen transfer coefficient in the bioreactor. Motioning the bioreactor, which is at least partially filled with culture medium, makes part of the carriers travel from a liquid phase, in which they are in contact with the culture medium, to a gas phase, in which they are not in contact with said medium. This increased oxygen transfer rate by at least 10 times compared to bioreactors of the prior art.

In a preferred embodiment, the bioreactor allows cells growth with density of from 50000 to 350 000 cell/cm² of carrier, preferably of from 100 000 to 250 000 cell/cm² of carrier, more preferably of from 150 000 to 200 000 cell/cm² of carrier depending on the cell type.

In a preferred embodiment, the bioreactor used in the method and/or the system of the invention is a small size bioreactor. Said bioreactor can be a circular bioreactor having a diameter of at least 10 cm, preferably at least 20 cm, more preferably at least 40 cm and of at most 50 cm, preferably at most 60 cm, more preferably at most 70 cm. Said bioreactor can also be a rectangular or square bioreactor having a height of least 10 cm, preferably at least 20 cm, more preferably at least 40 cm, even more preferably at least 50 cm, most preferably at least 60 cm and of at most 110 cm, preferably at most 100 cm, more preferably at most 80 cm, most preferably at most 70 cm. The width of said rectangular or square bioreactor is least 40 cm, preferably at least 50 cm, more preferably at least 60 cm and at most 100 cm, preferably at most 90 cm, more preferably at most 80 cm, most preferably at most 70 cm.

In a preferred embodiment, the system is implemented in a single portable chamber, suitable for a portable clean room such as a laboratory. Preferably, the system is implemented in small-scale cupboard which can be a portable chamber or portable clean room. Preferably, the dimensions of the small-scale cupboard are 0.8×1.6×1.8 m³. The system, according to any embodiment of the invention, provides for production of cells and cell derived products in a closed, self-sufficient environment. The functioning of said system requires minimal need for technician interaction. Integrating components, functions, and operations greatly reduces manpower and cost needed to produce a cells and/or cell-derived product. The integrated system reduces preparation and loading time and reduces the number of operator induced errors which can cause failure.

In a preferred embodiment, the system is provided with at least one withdrawal line 11 for withdrawing culture medium from the bioreactor. Said culture medium might be supplemented with grown cells and/or cells products. The withdrawal line comprises at least one sampling manifold 10 for collecting culture medium samples at any time during cells growth. Said samples are further analyzed thereby monitoring the evolution of cells growth.

The bioreactor of the system is fluidly connectable to at least one downstream unit which comprises different components or means suitable for further processing the supernatant, cultured cells and/or cells products. In a preferred embodiment, the downstream unit comprises pluggable means selected from the group comprising at least one filtration means, at least one harvest means, at least one dialysis means, at least one biomolecules purification means and at least one protein concentration unit or any combination thereof.

In a preferred embodiment, the downstream unit comprises at least one harvest means which is provided with at least one inlet and at least one outlet. Said means of the downstream unit is connectable to the cell culture unit of the system. The harvest means comprise at least one tubing for directing the collected supernatant to another component of the downstream unit. The harvest means further comprise at least on pump for withdrawing the supernatant from the bioreactor.

In a preferred embodiment, the downstream unit comprises at least one filtration means which is provided with at least one inlet and at least one outlet. Said means can be fluidly connected to the bioreactor or fluidly connected to the harvest means of the downstream unit. Preferably, the filtering means comprises a filter that will selectively retain molecules based on their mass in Dalton for instance. The filtration means might comprise virus hollow filters might be used to filter and remove virus particles from the supernatant. In this case, virus filtration works on the principle of size exclusion. When a protein solution with possible viral contamination is introduced into these hollow filters, the smaller proteins penetrate the filter wall and work their way to the outside of the filter while the larger virus particles are retained.

In a preferred embodiment, the downstream unit comprises at least one purification means which is provided with at least one inlet and at least one outlet. Said means can be fluidly connected to the bioreactor or fluidly connected to the harvest means or the filtration means of the downstream unit. Preferably, the purification means comprises at least one selection device. Said selection device can be a chromatography column such as such as affinity chromatography, ionic exchange chromatography (e.g. anion or cation), hydrophobic interaction chromatography, size exclusion chromatography (SEC), immuno-affinity chromatography which is a column packed with an affinity resin, such as an anti-IgM resin, a Protein A, a Protein G, or an anti-IgG resin. Anion exchange exploits differences in charge between the different products contained in the harvested supernatant. The neutrally charged product passes over the anion exchange chromatography column cartridge without being retained, while charged impurities are retained. The size of the column may vary based on the type of protein being purified and/or the volume of the solution from which said protein is to be purified.

The downstream unit can be customized depending on the needs of the user, and can be supplied with a combination of any of the aforementioned means. The user is hence provided with multiple end product possibilities: cells, filtered cells, filtered cells products, purified cells products or biomolecules. The user can choose and connect the different compartments of the downstream depending of the desired final product.

In a preferred embodiment, a waste collection container, into which metabolic wastes are being removed from the bioreactor, is provided. Said container is connected to the bioreactor and might be positioned inside the culture unit and/or inside the technical control unit of the system. Said container might also be provided outside the system while being connected to the bioreactor. In this case, the required connections for ensuring waste removal are known to the person skilled in the art.

In a second aspect, the present invention provides an integrated automated method for the production of cells and/or cell products comprising the steps of:

culturing cells in at least one bioreactor which is fluidly connected to a culture medium reservoir, said bioreactor being contained in a cell culture unit;
providing a mixture of at least two gases to the bioreactor; and
providing sterile ambient air into the cell culture unit;
wherein the bioreactor total volume is at most 1000 L.

In a preferred embodiment, the bioreactor total volume is at most 980 L, at most 960 L, at most 940 L, at most 920 L, at most 900 L, at most 880 L, at most 860 L, at most 840 L, at most 820 L, at most 800 L, at most 780 L, at most 760 L, at most 740 L, at most 720 L, at most 700 L, at most 680 L, at most 660 L, at most 640 L, at most 620 L, at most 600 L, at most 580 L, at most 560 L, at most 540 L, at most 520 L, at most 500 L, at most 490 L, at most 480 L, at most 450 L, at most 420 L, at most 400 L, at most 380 L, at most 350 L, at most 340 L, at most 330 L, at most 320 L, at most 310 L, at most 300 L, at most 290 L, at most 280 L, at most 270 L, at most 260 L, at most 250 L, at most 240 L, at most 230 L, at most 220 L, at most 210 L, at most 200 L, at most 190 L, at most 180 L, at most 170 L, at most 160 L, at most 150 L, at most 140 L, at most 130 L, at most 120 L or at most 100 L, or any value comprised in between the aforementioned values.

In a preferred embodiment, the bioreactor total volume is at least 0.5 L, preferably at least 1.5 L, preferably at least 3 L, more preferably at least 5 L, even more preferably at least 10 L, most preferably at least 20 L, even most preferably at least 30 L. Preferably, the bioreactor total volume is at least 40 L, at least 50 L, at least 60 L, at least 70 L, at least 80 L, at least 90 L or any value comprised in between the aforementioned values. The bioreactor total volume and the bioreactor itself are small compared to the conventional bioreactors used for cell culture. This is advantageous in terms of required space for the system and for ease of use.

In a preferred embodiment, the method of the invention is suitable to be carried out by a system as described above and according to any embodiment of the present invention.

Preferably, the culture medium volume provided to the bioreactor for culturing cells is sufficient to fill at least about half of the bioreactor expression volume. By bioreactor expression volume reference is made to the bioreactor volume used for the expression of available surface. For instance, if the bioreactor total volume is 300 L and its expression volume is 10 L, then about 5 L of culture medium are provided to the bioreactor while the remaining volume of about 295 L is circulating between the bioreactor and the culture medium reservoir.

In a preferred embodiment, the bioreactor is motioned or moved during cell culture. Said motioning or movement is selected from movements from right to left, up to down, rotating along a horizontal axis, rotating along a vertical axis, a rocking motion along a tilted or inclined horizontal axis of the bioreactor or any combination thereof. Said movement or motioning might be performed in a continuous or discontinuous mode.

Preferably, the bioreactor wherein cell are grown is provided with carriers selected from the list comprising fibers, microfibers, hollow microfibers, hollow filter, tangential flow filter, settler, microcarriers, microcarriers containing stirred vessels or any combination thereof. Said carriers provide for an excellent substrate for the cells to grow on. Moving or motioning the bioreactor allows getting the cells loose from the carrier—prior harvesting cells from the bioreactor for instance.

In a preferred embodiment, a predetermined temperature and/or a predetermined pressure is maintained constant in the cell culture unit. Said predetermined pressure is of about −2 to −5 mbar, preferably −3 to −4 mbar. By preference, the predetermined temperature of the cell culture unit is between 20° C. and 40° C., more by preference between 25° C. and 37° C. The operating temperature of the downstream unit may be between 0° C. and 25° C., more preferably between 1° C. and 20° C., even more preferably between 2° C. and 10° C., most preferably about 4° C. The temperature of both units is maintained by cooling and/or warming units and maintenance of the temperature may be checked by sensors.

By preference, the method of the current invention further comprises the steps of growing cells to a density at least 50 million cells per ml and fluidly connecting the cell culture unit and/or the bioreactor to a downstream unit. Preferably, at least one sensor is provided for measuring the cell density inside the bioreactor. Preferably, the bioreactor allows high density cell growth. Said density is of at least 80 million cells/ml, more preferably at least 100 million cells/ml, most preferably at least 200 million cells/ml. Said density can reach 600, 500, 400 or 300 million cells/ml.

In a preferred embodiment, bioreactor's supplemented medium is transferred from the bioreactor to or harvested into the downstream unit. Said bioreactor and downstream unit are fluidly connected to each other. A pump might be provided for transferring the supernatant into the downstream unit.

The downstream unit may comprise filtration means and/or harvest means and/or dialysis means and/or biomolecules purification means such as proteins or peptides purification. In its most simple form, said downstream unit comprises solely means for harvesting the desired end-product, without any prior filtration/purification/dialysis steps. The components of the downstream unit are easily connected to or disconnected from said unit and can hence be easily replaced, cleaned or sterilized. The downstream unit can be customized depending on the needs and desires of the users, and can be supplied with a combination of any of the aforementioned units. The user is hence provided with multiple end product possibilities, cells, filtered cells, filtered cells products, purified cells products or biomolecules. The user can choose and connect the different compartments of the downstream depending of the desired final product.

In a preferred embodiment, the downstream unit receives supplemented medium or medium supplemented with biomolecules from said bioreactor in continuous mode. Preferably, the downstream unit receives at most 1000 ml/min of medium supplemented with biomolecules from said bioreactor in continuous mode. Preferably, the transfer of the supplemented medium is initiated when a predetermined cell density is reached inside the bioreactor. Said predetermined cell density is at least 30 million/ml, preferably 40 million/ml, more preferably 50 million/ml, most preferably 60 million/ml. In a preferred embodiment, in parallel to the transfer of the supplemented medium from the bioreactor to the downstream unit, culture medium is added from the internal culture medium tank to said bioreactor such as to maintain the initial volume of culture medium in the bioreactor. For instance, if at the start of the process the bioreactor contained 80 L of culture medium, once the transfer of supplemented medium from the bioreactor to the downstream unit is initiated, new culture medium is added to the bioreactor in sufficient volume such as to maintain a volume of 80 L in said bioreactor. If the transfer of supplemented medium from the bioreactor to the downstream unit is performed in continuous mode, the addition of new culture medium from the internal culture medium tank into the bioreactor will be also carried out in continuous mode. The method and the system of the present invention thereby allow the treatment of the supplemented culture medium in the downstream unit in parallel to the growth of the cells in the bioreactor. This provides several advantages compared to processes wherein cells are grown in large bioreactors containing large cell culture volumes followed by stopping said cell culture after a certain time period or when reaching a certain concentration and then starting the downstream processes of the large volume of cell culture. Amongst the advantages we can mention a considerable yield increase and thereby a considerable cost decrease.

In a preferred embodiment, the medium supplemented with biomolecules received by the downstream unit undergoes at least one process selected from the group comprising filtration, harvesting, dialysis, biomolecules purification and protein concentration or any combination thereof.

The supplemented medium is preferably harvested in a continuous way at a predetermined small volume rate. Said volume rate is of at least 100 ml/min, preferably at least 150 ml/min, more preferably at least 200 ml/min, most preferably at least 250 ml/min. Said volume rate is at most 1000 ml/min, preferably at most 800 ml/min, more preferably at most 600 ml/min, most preferably at most 400 ml/min. The supernatant harvest can also be performed in a discontinuous way. The harvested supernatant might then be subject to a subsequent treatment selected from simple harvesting, filtering, molecules purification, storage or any combination thereof. The treatment of small volumes of supplemented medium considerably reduces yield loss and improves the treatment quality and efficiency, e.g. better filtration and/or the purification quality. In addition, no scaling up of the operations carried out in the downstream unit is required thereby avoiding spending time and money for scaling up said operations. The continuous harvest mode can be initiated by the operator based on product concentration. The harvest continues until a pre-programmed time interval has passed or until the operator manually terminates the harvesting using a user's interface provided in the system of the invention.

In a preferred embodiment, non-disrupted cultured cells are harvested in bulk from the bioreactor into a bag provided in the downstream unit. The cells can be hybridoma cells, transfected or transduced cells or stably transfected cultured cells. In order to get the cells loose from their substrate (the fibers), the bioreactor may be subjected to a discontinuous or a continuous agitation prior to harvesting. Said agitation is from 10 to 150 Hz at amplitude 1-5 mm, preferably from 20 to 100 Hz at amplitude 1-5 mm. In the event the bioreactor is provided with carriers, the agitation will separate the cells from said carriers and bring them into the supernatant. Harvesting of the supernatant is performed using harvest means comprising at least one pump. The bag and/or the downstream unit can be adapted to maintain the harvested supernatant at the same temperature as the temperature of the culture medium or at a different temperature. The harvested cells might be maintained in the bag of the downstream unit at a temperature of about 4° C. The cultured cells harvested in bulk can be filtered using filtering means of the downstream unit prior directing said cells into the bag.

In a preferred embodiment, cultured cells are infected and subsequently disrupted/lysed in a thereto designed location in the downstream unit. The supernatant comprising the cell debris and the desired products is then harvested using harvest means from the bioreactor. Harvesting rates are as mentioned above. The supernatant can be harvested and stored for further use into a bag provided in the downstream unit as mentioned above. The harvested supernatant might be subject to a filtration using filtration means prior to storage into a bag of the downstream unit. Alternatively, the collected supernatant can be filtered and/or subject to a purification step for separating a specific molecule, such as an antibody, from said supernatant.

Purification can be performed using purification means of the downstream unit. Said means can be automated means for obtaining a purified biological product such as proteins, urified antibodies rom the supernatant. In a preferred embodiment, the purification means comprise at least one or any combination of the following: a selection device such a purification chromatography column (affinity purification, ion exchange, etc.), a sequence of purification columns or membrane absorbers at least one liquid reservoir, a device for flowing liquid from the reservoirs and into the selection device, a device for diverting the effluent from the selection device. The purification means are capable of being installed into the small-scale cupboard-sized system of the invention via a single motion or “snap-on” or “quick-load” technique and comprises mechanical and electrical interfaces for communicating with the other components of the system of the invention. It is to be understood that the required buffers and solutions for performing the purification process or step might be provided in at least one bag. Said bag can be positioned inside or outside the downstream unit and is naturally provided with the necessary connections to ensure its connection with to the purification unit.

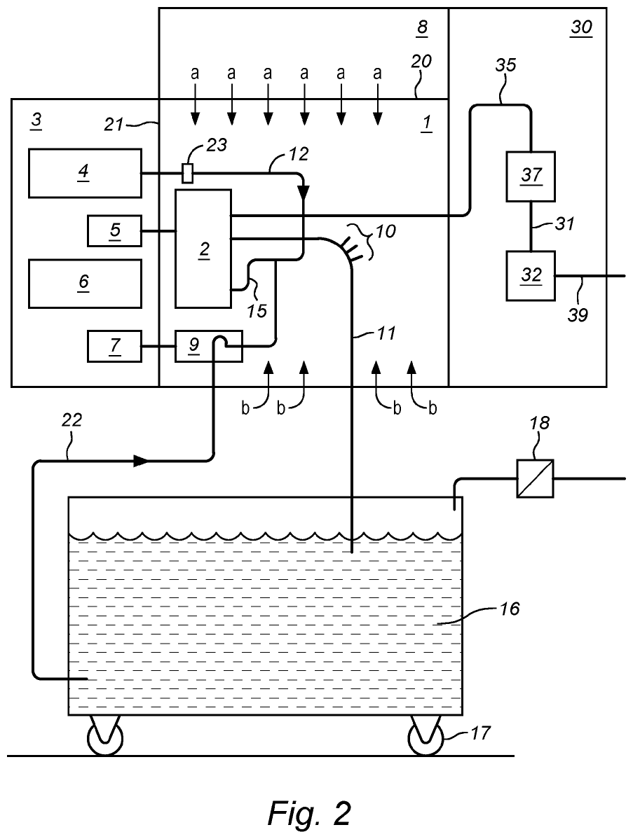

FIG. 2 shows an embodiment of the system which is adapted for harvesting, filtering and purifying at least one cell product, such as a protein or a peptide. The cell culture unit 1 is fluidly connected to the downstream unit 30 through the out-tubing 35. The culture unit 1 is as described above. The out-tubing 35 directs the supernatant to a filtering means 37. A pump, or harvest means, might be provided for collecting the supernatant of the bioreactor. The pump can be programmed such as to start the supernatant collection from a pre-defined time period from the start of the culture. The pump can be programmed such as to collect a pre-fixed volume of supernatant in an automated continuous mode. The collected filtered supernatant is then directed to a purification means 32 of the downstream unit 30 via at least one tubing 31. The obtained purified cell product can be stored in a tank connected to the purification means or directed, via at least one tubing 39, to another component of the downstream for further applications or to simply be collected by the user.

In a preferred embodiment, the purification means, e.g., an affinity column, and/or the filtration means are connected to multiple liquid reservoirs. The reservoirs each contain liquid, such as a wash buffer, an elution buffer, or a neutralization solution, for delivery to the purification means and/or the filtration means. The purification means further comprise pre-sanitized or pre-sterilized device for flowing liquid from the reservoirs into the chromatography column for instance. For example, pre-sterilized valves and tubing which connect the reservoirs to the column might be used.

Purification using a chromatography column is known to the person skilled in the art and can be performed using the adequate buffers for eluting the desired biomolecules. Upon eluting the desired biomolecule, the eluted purified protein can be automatically deposited into a pre-sterilized, disposable collection vessel provided in the downstream unit and removed from the purification means. Alternatively, the eluted purified protein can undergo further automated processing. A purified protein, e.g., antibody, is substantially free from host cell contaminants such as host cell proteins, nucleic acids and endotoxins.

In a preferred embodiment, the eluted protein is transferred to different solutions. The transfer occurs automatically using a pre-sterilized diafiltration module. Diafiltration is the fractionation process that washes smaller molecules through a membrane and keeps molecules of interest in the retentate. Diafiltration can be used to remove salts or exchange buffers. In discontinuous diafiltration, the solution is concentrated, and the lost volume is replaced by new buffer. Concentrating a sample to half its volume and adding new buffer four times can remove over 96% of the salt. In continuous diafiltration, the sample volume is maintained by the inflow of new buffer while the salt and old buffer are removed. At least 99% of the salt can be removed by adding up to seven volumes of new buffer during continuous diafiltration. Specifically, the diafiltration module is used to further purify the protein (e.g., the antibody) and uses the tangential flow filtration principle whereby molecules over 50,000 Daltons (e.g., the antibodies, such as IgG and IgM) cannot pass through the membrane but small molecules, such as buffers, can pass through. Accordingly, the diafiltration module can be used to exchange one buffer for another and is a more efficient substitute for dialysis. Diafiltration can be used to neutralize pH and as a concentration step (to concentrate the cell product).

In a preferred embodiment, the harvest means and/or the filtration means and/or the purification means include at least one monitoring device for monitoring the circulating medium:

non-filtered harvested supernatant, filtered supernatant, purified and eluted product, etc. The monitoring device can be a probe or sensor for measuring the conductivity and/or the pH and/or absorbance at a particular wavelength of said circulating medium. One or more pressure sensors may be included for monitoring circulating medium pressure for excessive pressures, or for control of pump speed, e.g., to maintain the pump speed of the harvest means for instance at a desired pressure.

In a preferred embodiment, the system is adapted to the desired product. This means that if cells in bulk are to be provided, the system will comprise a downstream unit in which at least one collection bag is provided. If filtered cells are to be provided, the system will comprise the cell culture unit and the downstream unit in which filtering means and at least one collection bag are provided. If a specific protein is to be provided, the system will comprise the cell culture unit and the downstream unit in which at least filtering means and purification means are provided.

In a preferred embodiment, the method and the system of the present invention are devoid of closed loops or recirculation loops. This means that the supplemented culture medium is not returned to the bioreactor at any stage of the process such as after its passage through the downstream unit. This is advantageous as it considerably reduces contamination risks. Furthermore, this simplifies the setup and the installation of the system thereby reducing costs.

In a preferred embodiment, the method is fully controlled by a programmable controller. This considerably limits human intervention thereby considerably reducing errors and contamination risk. The operator might initiate some actions of the cell culture unit and/or the downstream unit such as the culture process and/or harvesting process and/or the purification process through a user interface such as a touch screen interface on portable chamber and/or the cell culture unit.

In a preferred embodiment, cells (mammalian or insect cells) and adapted culture medium are introduced in the bioreactor. Adapted culture medium refers to the composition of the medium which is required for the growth of the cells. Said compositions are known to the person skilled in the art and generally comprise salts, vitamins, amino acids, sugars or any combination thereof. The culture medium is preferably provided to the bioreactor from an external culture medium reservoir, i.e. not contained in the system of the invention. Preferably, the culture medium is preheated prior being provided to the bioreactor. The preheat temperature of the culture medium is of from 20 to 40° C., preferably from 25 to 38° C., more preferably from 30 to 37° C. In a most preferred embodiment, said culture medium is pre-heated at about 37° C.

In a preferred embodiment, cells are cultured in the bioreactor for a time period which can vary from few hours to several days depending on the cultured cells. The culture time period is at least 4 hours, at least 10 hours, at least 24 hours, at least 5 days at least 7 days or any time comprised in-between. The culture time period is at most 70 days, at most 60 days, at most 50, at most 40 days, at most 30 days, at most 20 days, at most 10 days or any time comprised in-between.

Depending on the final product, viral transduction or introduction of viral vectors can be used. Viral replication competent vectors or replicons have been used for a long time as an alternative expression system to increase the yields of therapeutic proteins in mammalian cells. The target gene(s) can be expressed under transcriptional control of viral promoters whereby the mRNAs accumulate to extremely high levels in the cytoplasm after transfection and upon replication, yielding large amounts of target protein. The viral infection can lead to a transduction process without lysis of the cultured cells or to the lysis of the cultured cells thereby bringing the cells content into the culture medium of the bioreactor. Alternatively, hybridoma cells or stably transfected cells can be cultured in order to produce the desired protein or peptide such as an antibody or an antibody fragment.

The method and/or the system of the present invention can be used for the culture of any cell line and/or for the production of any desired protein and peptide. Examples of preferred cells used in the current system include but are not limited to the Vero cells, the CHO cells, Hek293T cells, COS cells, 293T cells, HeLa cells, Hep-2 cells, MCF-7 cells, U373 cells or any other cell line. Examples of viral replication systems include but are not limiting to polyoma viruses, lentiviral systems, retroviral systems, adenoviral systems, adeno-associated viruses.

In a preferred embodiment of the current invention, bioreactor's supplemented medium is transferred from the bioreactor to or harvested into a downstream unit. Said downstream unit is positioned outside the system of the invention and might have a wall in common with any unit of the system, preferably with the cell culture unit. It is to be understood that the bioreactor and the downstream unit are fluidly connected to each other. A pump might be provided for transferring the supplemented medium into the downstream unit. Supplemented culture medium, also herein called supplemented medium, refers to the culture medium of the bioreactor which might comprise cultured cells and/or their products. Cells product refers to biomolecules such as proteins, peptides, produced by the cells and/or any other cell biomolecules derived from cell membrane lysis.

In a preferred embodiment, at least one supply means 7,9, connected to the bioreactor 2 and to a culture medium reservoir 16, ensures that the bioreactor 2 is provided with culture medium (FIG. 1). The culture medium transfer might be performed continuously and/or at a constant rate and/or at variable rates. Said medium transfer can also be performed discontinuously and/or at a constant rate and/or at variable rates.

In a preferred embodiment, the method further comprises the step of measuring physical and/or chemical parameters of the cell culture and/or culture medium. Said parameters are selected from the group comprising temperature, pH, salinity, acidity or any combination thereof. Measurements can be performed on samples taken from the culture medium before being injected into the bioreactor and/or from culture medium taken from the bioreactor during cells growth which might comprises cells and/or cell products. Said sampling might be performed using the manifolds of the system.

In a preferred embodiment, the method and/or the system of the present invention are used for viral vaccine production. For this purpose, preferably adherent cells are used and gown in a bioreactor packed with any entrapment system, preferably microfibers. The packing preferably allows about at least 0.5 m² of growth surface per L of bioreactor or any of the aforementioned growth surface values. The cells grow up to at least 100 000 to 250 000 cells/cm² depending on the cell type. The preferred volumes of used culture medium are of about 0.3 ml of medium/cm² for cell growth and 0.3 ml of medium/cm² for viral production. Examples of bioreactor volumes and surfaces are summarized in table 1 below. It is to be understood that any possible combination of values from table 1 are also comprised in the present application.

TABLE 1
Examples of bioreactor vulture medium volumes and
growth surfaces for viral vaccine production

| | Culture medium | | |
|---|---|---|---|
| | introduced | | |
| Bioreactor | in the bioreactor | Growth surface | Bioreactor |
| expression | expression | in the bioreactor | total |
| volume in L | volume in L | in m² | volume in L |
| 10 | 5 | 100 | 300 |
| 8 | 4 | 80 | 240 |
| 5 | 2.5 | 50 | 150 |
| 2 | 1 | 20 | 60 |
| 1 | 0.5 | 10 | 30 |
| 0.1 | 0.05 | 1 | 3 |
| 0.05 | 0.025 | 0.5 | 1.5 |

In a preferred embodiment, the method and/or the system of the present invention are used for antibodies production. For this purpose, preferably suspension and/or adherent cells are used and gown in a bioreactor packed with microfibers or comprising any other entrapment system. The packing preferably allows about at least 0.5 m² of growth surface per L of bioreactor or any of the aforementioned growth surface values. The cells grow up to at least 100×10⁶/ml of bioreactor depending on the cell type. Preferably, the bioreactor is supplemented with a volume of culture medium every day. Said volume is preferably up to 1.5 times the initial culture medium volume introduced in the bioreactor. The process can be performed up to 30 days. Examples of bioreactor volumes and surfaces are summarized in table 2 below. It is to be understood that any possible combination of values from table 2 are also comprised in the present application.

TABLE 2
Examples of bioreactor culture medium
volumes for antibodies production

| Culture medium | Maximum culture medium | |
|---|---|---|
| introduced in the | volume introduced in the | Bioreactor total |
| bioreactor in L | bioreactor in L and per day | volume in L |
| 10 | 15 | 450 |
| 8 | 12 | 360 |
| 5 | 7.5 | 225 |
| 2 | 3 | 90 |
| 1 | 1.5 | 45 |
| 0.1 | 0.15 | 4.5 |
| 0.05 | 0.075 | 2.25 |

The method and/or the system of the invention provide for the production of monoclonal antibodies, recombinant proteins or any other cell secreted biologic molecules. Viral vaccine production and antibodies production using the method and/or the system of the present invention, and in particular using the above mentioned culture medium volumes and growth surfaces, allows (i) to carry out the production process at small and intermediate scale and to implement clinical and/or commercial productions of vaccines, gene vectors or oncolytic viruses. For instance for antibodies, small scale production of from 10 to 50 L and intermediate production of about 250 L can be achieved using the system and/or the method of according to any embodiment of the invention.

The method and/or the system according to the invention are particularly useful for the production of biosimilar antibodies. The term ‘biosimilar’ antibodies is to be understood as ‘generic’ versions of ‘originator’ antibodies which have the same amino acid sequence as those ‘originator’ antibodies but which are produced from different clones and/or by different manufacturing processes.

In a preferred embodiment, the method and/or the system of the present invention allows production of vaccines quantities of about 940 roller bottles of 850 cm² which for a majority of vaccines a commercial production scale. In a preferred embodiment, the method and/or the system of the present invention allows production of antibodies quantities up to 360 g, preferably up to 400 g which is also for a majority of antibiotics a commercial production scale.

The method and/or the system can be used for the production of:

Anti-inflammatory biomolecules or any antibody such as infliximab, adalimumab, basiliximab, daclizymab, omalizumab, palivizumab and abciximab;
Anti-cancers biomolecules such as gemtuzumab, alemtuzumab, rituximab, transuzumab, nimotuzumab, cetuximab, bevacizumab;
Human vaccines such as but not limited to polio vaccine (IPV), Rotavirus vaccine, Influenza vaccine, Yellow Fever vaccine, Varicella vaccine, Measles, Mumps, Rubella, Hepatitis and Rabbies vaccine;
Veterinary Vaccines such as but not limited to Marek vaccine and Newcastle vaccine. The methodology and system can also be used for the production of RSV-antibody based vaccine;
and formulations thereof.

The person skilled in the art will appreciate that necessary tubing and/or pumps can be provided within the system for achieving the required connections between the different units and/or means of the system. Further, the system can be provided with a plurality of switch valves used to route the fluids between said different compartments. In addition, a software program for running the system and/or the method according to an embodiment of the invention can be provided.

Although the present invention has been described with reference to preferred embodiments thereof, many modifications and alternations may be made by a person having ordinary skill in the art without departing from the scope of this invention which is defined by the appended claims.

What is claimed is:

1. An integrated system for the production of cells or cell products, comprising:
at least one cell culture unit comprising at least one bioreactor for culturing cells, wherein said at least one bioreactor total volume is at most 1000 L;
at least one technical control unit comprising a controller for controlling a plurality of cell culture parameters of the at least one bioreactor, wherein said at least one technical control unit is fluidly connected to said at least one cell culture unit; and
at least one pump for providing the bioreactor with culture medium from a reservoir, the pump including a motor and a pump head, wherein the motor is positioned in the at least one technical control unit while the pump head is positioned in the at least one cell culture unit.

2. The system according to claim 1, wherein the at least one technical control unit is adapted for controlling movement of the bioreactor.

3. The system according to claim 1, further including a gas source fluidly connected to the bioreactor, said gas source including a mixer for mixing at least two different gases.

4. The system according to claim 1, wherein a predetermined temperature or predetermined pressure is maintained constant inside the at least one cell culture unit.

5. The system according to claim 1, wherein the at least one bioreactor comprises microfibers, a hollow filter, a tangential flow filter, a settler, or any combination thereof.

6. The system according to claim 1, further including at least one fluid processing unit downstream of the at least one cell culture unit, the at least one fluid processing unit comprising at least one filter, at least one harvester, at least one purifier, at least one concentrator or any combination thereof.
